(19) United States
(12) Patent Application Publication
(10) Pub. No.: US 2018/0325696 A1
(43) Pub. Date: Nov. 15, 2018

(54) Holder For An Acetabular Cup Implant

(71) Applicant: Embody Orthopaedic Limited, London (GB)

(72) Inventor: Robert Wozencroft, Epsom Surrey (GB)

(21) Appl. No.: 15/774,222

(22) PCT Filed: Nov. 7, 2016
(86) PCT No.: PCT/GB2016/053475

§ 371 Date: May 7, 2018

(30) Foreign Application Priority Data
Nov. 6, 2015 (GB) 1519626.4

(51) Int. Cl. A61F 2/46

(52) U.S. Cl. CPC A61F 2/4609; A61F 2002/30331

(57) ABSTRACT

A holder for an implantable acetabular cup implant, said holder comprising a body portion and at least one fastening means attached to the body portion which is operable in order to secure said holder to said implantable device.

### BACKGROUND

Acetabular cup implants with large diameter bearings, such as resurfacing cups or large diameter total hip replacements (THR's), are a particular challenge to hold during implantation. The surgeon must forcibly impact the cup into a prepared, slightly undersized bone socket during fitting, whilst ensuring that the cup is implanted in the correct orientation which is critical to the function of the hip implant. Furthermore, it is essential to protect the bearing surface from damage during this forcible impaction process. Most conventional THR cups consist of an outer shell and separate bearing liner which is inserted after the metal shell is implanted. Therefore the inside of the metal shell is used to provide holding features such as a screw thread or bayonet attachment. However this area is not available on resurfacing cups or cups with pre-fitted liners because the inside surface forms the bearing surface. Furthermore, the outer surface is usually fully embedded in the bone socket, so only the cup rim is accessible. Previous resurfacing designs have attempted to solve this problem in various ways, but these have resulted in compromises to the designs, either by reducing bearing arc, or reducing outer fixation surfaces or with the presence of holding features that caused snagging and aggravation of the soft tissue structures such as Psoas tendon in close proximity to the cup rim. It is now recognised that attempts by manufacturers to provide adequate cup holding solutions have a serious, deleterious effect on the clinical outcome of many resurfacing designs which have now been withdrawn from clinical use partly due to these problems.

A new generation of resurfacing implants and large diameter THR's utilising non-metal bearing materials including zirconia toughened alumina ceramic (ZTA) and cross linked ultra-high molecular weight polyethylene (UHMWPE) pose an even bigger challenge for cup holding during implantation. In the case of ZTA, due to the restraints of the manufacturing process and hardness of the material, it is technically difficult and very expensive to add small holding features that may be possible (if undesirable) in metal. Also in the case of UHMWPE, it is critical for wear characteristics and material strength that full wall thickness is maintained, so holding features that reduce wall section would be undesirable. In addition, some new generation devices are intended for specific rotational alignment of asymmetric features on the cup which also add difficulties for cup holding.

### STATEMENT OF INVENTION

To overcome these difficulties, the present invention provides a cup holding solution without the need for special holding features on the cup and, furthermore, fully protects the implant bearing surface during forcible insertion.

### DESCRIPTION & ADVANTAGES

Preferably, the holder is manufactured from one piece. A preferred material to manufacture the holder from is a plastics material. Preferably, the holder is single use. The holder may be supplied with the implant or separately with the instruments. Preferably, the holder is manufactured in plastic (such as nylon) which will not damage the hard implant bearing surfaces. Preferably, the holder is manufactured by an additive manufacturing process. The skilled person is aware of a number of different types of additive manufacturing processes, for example selective laser sintering (SLS) or stereolithography (SLA). Alternatively the holder could be moulded in plastic, for example by injection moulding or cold curing casting resin. Alternatively the holder could be manufactured in plastic by any other means, including machining.

Securement of the holder to the cup implant is achieved with fastening means. Such fastening means are preferably clips. In a preferred embodiment, the fastening means (e.g. clips) are integrally formed with the holder itself. Alternatively, the fastening means (e.g. clips) may not be formed with the holder, but may be attached to the holder at a later stage during the manufacturing process. Preferably, the holder is manufactured in one piece with resilient connections to the fastening means (e.g. clips) to prevent dis-association of the clips from the body of the holder.

The clips are arranged such that they are able to press down over the outside surface of the cup implant where a surface of part of each clip is able to contact the outside surface of the implant. The clips are able to maintain a pinching grip between holder and cup implant. This is particularly advantageous in embodiments where the holder and fastening means are made from a material having a resilient nature. The holder may be supplied separately and assembled onto the cup implant intra-operatively or it may be pre-assembled with the cup implant in the same sterile pack, for example a blister pack. If supplied with the implant, preferably the fastening means (e.g. clips) will be in the manufactured position (not pressed down into the secured position) so that the surgeon can easily inspect the bearing surface prior to implantation, however alternatively the clips may be in the secured position. Preferably, there are two fastening means (e.g. clips) but there could be up to six. In some embodiments, there are 1, 2, 3, 4, 5 or 6 clips. Preferably the fastening means (e.g. clips) are spaced equally to provide a uniform grip on the cup. However the position of the clips is not critical because they do not rely on mating holding features on the cup, so they could be positioned in any location relative to the cup rim (e.g. unequally spaced around the cup rim). In a preferred embodiment the cup has a contoured rim, therefore the holder and clips are located in a specific rotational position, however the clips could be located elsewhere on the cup rim.

In an alternative embodiment, it may be desirable for the cup to have a mating feature or features to accept the clips (for example recesses). These could provide additional resistance to rotation or additional grip between the cup implant and the holder. Alternatively in the case of a traditional acetabular cup with planar face and without asymmetric contours on the rim, the mating feature(s) to accept the clips may provide rotational orientation, or additional resistance to rotation, or additional grip or all three of these things.

Preferably, the clips self-release as they are pushed off against the rim of the socket. However if they do not self-release due to absence of bone at their location, the holder and shaft can also be released by gently pulling them away from the cup when it is fixed in the bone socket. In an alternative embodiment, one or more of the fastening means might have pads which extend away from the body of the holder. These can provide a means of manually releasing the clips from the implant by pressing the pads together towards the centre line. Also in addition or in an alternative embodiment, there is provided a latching member on the surface of the fastening means which interacts with a recess on the inner surface of the holder. Such a latching means is able to lock the clips in the secured position.

Once the holder is secured to the cup implant via the fastening means (e.g. clips), a shaft is inserted into the holder to allow positioning and impaction of the cup into the bone socket. Alternatively, the shaft can be inserted into the holder before the holder is secured to the cup implant. Preferably, the shaft connection feature has a slight press fit into the holder so they remain fixed together once assembled. Alternatively, a reversible snap fit or a snap fit that requires releasing by the user may be used. During insertion into the bone socket, the bearing surface remains covered at all times for protection. This is advantageous as it prevents, or significantly minimises, the risk of accidentally compromising the bearing surface of the cup implant during the insertion stage. Preferably, the holder fits exactly to the contoured cup rim providing rotational control between cover and cup implant. Preferably, the shaft also has a rotation indexing feature ensuring rotational alignment to the holder and cup, so the cup can be rotationally orientated as intended in the bone socket. In addition to the shaft, a cup alignment guide (which might be patient specific) may be employed to position the cup in the bone socket in accordance with a pre-operative bone scan and predetermined plan. In embodiments where the opposite end of the shaft also has a rotation indexing feature, the alignment guide can direct the cup implant into the planned inclination angle, anteversion angle and rotational position. Preferably, the shaft is made from metal. However, the skilled person will be aware of a number of different materials that can withstand the force of impacting the shaft during the operative process in order to fix the cup implant in position.

### INTRODUCTION TO DRAWINGS

An example of the invention will now be described by referencing to the accompanying drawings:

FIG. 1 is an exploded view showing a holder and cup implant.

FIG. 2 is a cross section close-up of the assembled holder and cup implant of FIG. 1 showing the integral clip in the manufactured position.

FIG. 3 is a cross section view of the assembled holder and cup implant of FIG. 1 with the clips in the manufactured position.

FIG. 4 is a cross section view of the assembled holder and cup implant of FIG. 1 with the clips in the pushed down/secured position.

FIG. 5 is an exploded view showing insertion of the shaft into the assembled holder and cup implant of FIG. 1.

FIG. 6 shows the fully assembled holder, cup and shaft of FIG. 5 as the cup is being inserted into the bone socket.

FIG. 7 shows the holder of FIG. 6 with shaft attached being withdrawn, having been disconnected from the cup which is fully inserted in the bone socket.

FIG. 8 is an alternative embodiment, cross section view of an assembled holder and cup implant with the clips in the manufactured position.

FIG. 9 is an alternative embodiment, cross section view of an assembled holder and cup implant with the clips in the secured position.

FIG. 10 is a close up of FIG. 8 showing the clip latch in manufactured position.

FIG. 11 is a close up of FIG. 9 showing the clip in the pushed down/secured (latched position).

### DESCRIPTION WITH REFERENCE TO DRAWINGS

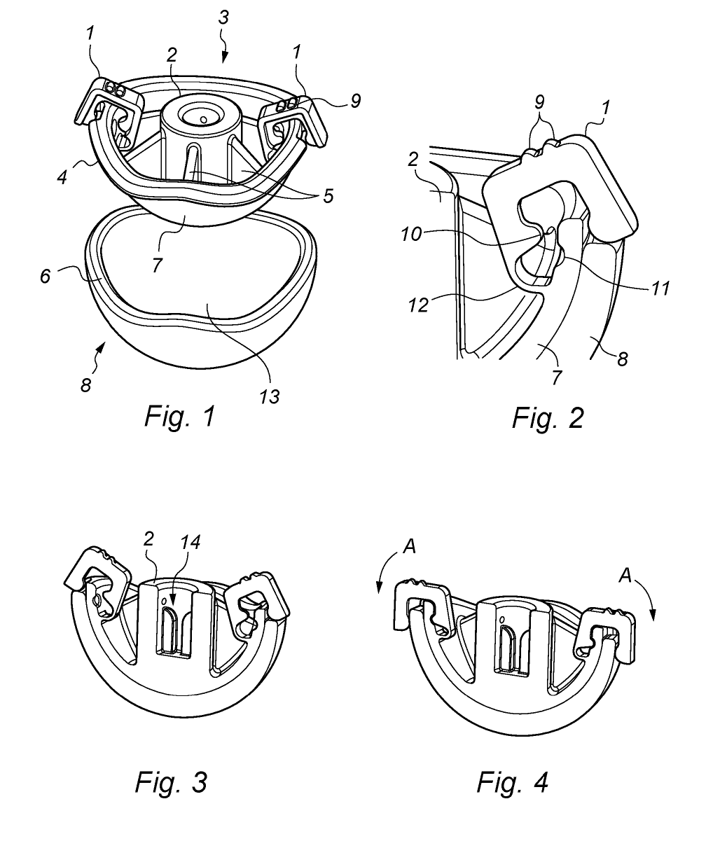

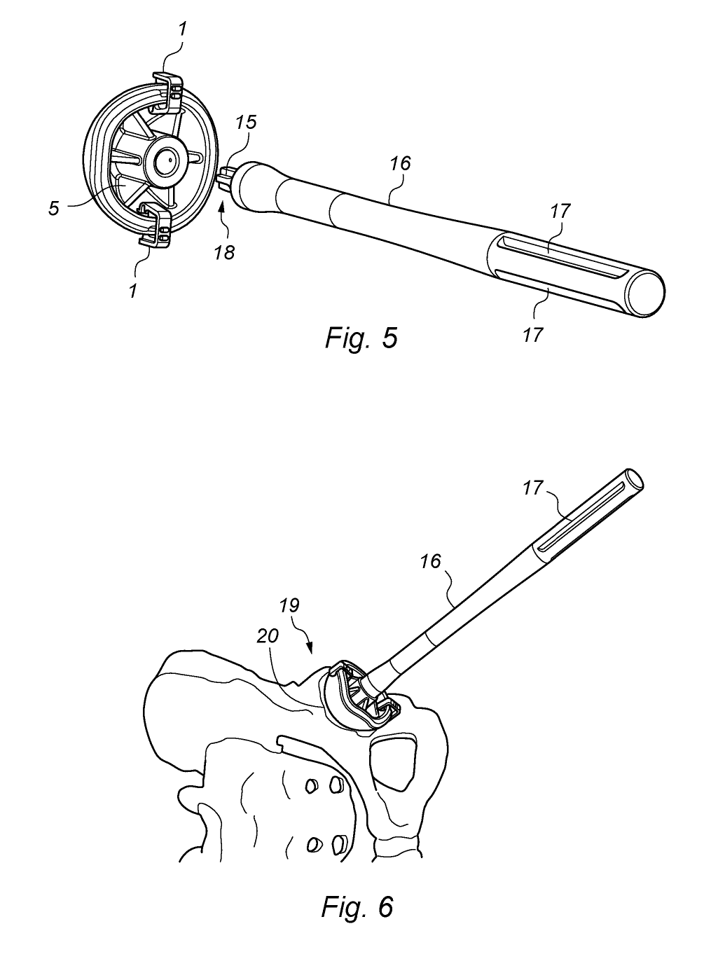

FIG. 1 shows the cup implant (8) with bearing surface (13) and holder (3) above. The integral clips of the holder are in their manufactured position and it is also shown that the contoured rim of the cup (6) matches the contoured flange of the holder (4). In some embodiments, however, it is not necessary that the contoured flange of the holder follows precisely any contour that may be present on the rim of the cup implant. A central boss (2) accepts the shaft (16) via shaft connection (18) shown in later FIGS. 5-7.

In some embodiments, it can be advantageous to have one or more fins (5) e.g. 1, 2, 3, 4, 5, 6, 7, or 8 emanating from the central boss and arranged to be secured onto the inside surface of the holder. These are able to provide support to the central boss itself on the impaction of the holder during surgery, thereby reducing the risk of distortion of the central boss. Moreover, the fins can act to distribute the force of the impaction equally, or as desired if the fins are not equally distributed, throughout the holder which in turn transmits the impaction force to the cup implant.

FIG. 2 shows the resilient connection (12) between clip (1) and holder body (7). The resilient connection (e.g. a bridge, or hinge) is preferably a relatively thin piece of material that connects the clip to the holder body. As the bridge is thin it is able to flex and bend, therefore allowing the clip to move in relation to the holder body such that a surface of the clip can extend over and interact in a gripping fashion with the outside of the cup implant.

Visible in FIGS. 1-4 are the surface grips (9) to press to secure the clips to the cup implant (8) and the engaging features (10 & 11) to guide the clips in the secured position when assembled on the cup implant. Although the FIGS. show surface grips (9), in alternative embodiments such grips are not required and they may be absent from the clips. Likewise, although the FIGS. show engaging projections (10) which fit into recesses (11) in the holder, these are not an absolute requirement and may be omitted in some configurations of the holder. Visible in FIGS. 3 & 4 is the female rotational indexing feature (14) which accepts the corresponding male indexing feature (15) on a shaft (16) in a single aligned rotational position. The shaft is used for impacting the holder and implant arrangement into the hip bone. FIGS. 5 & 6 shows the opposite end rotational indexing feature (17) which mates with an optional cup alignment guide (not shown).

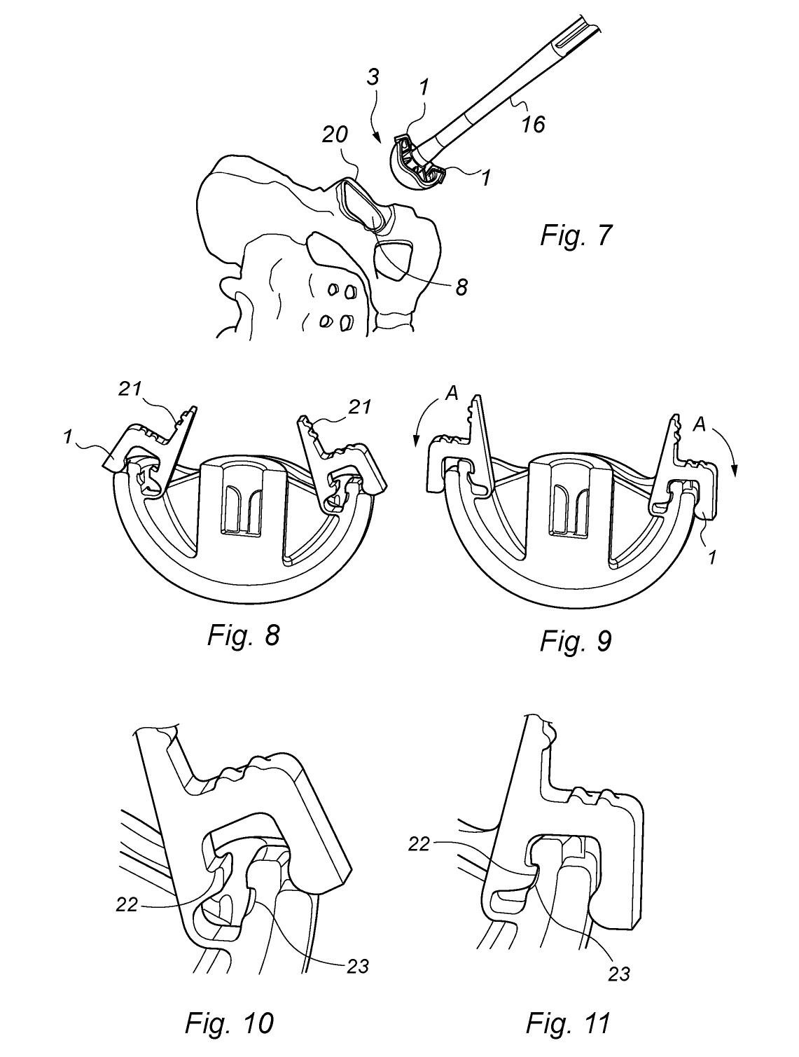

FIG. 6 shows the cup being inserted into the bone socket (19), just before the point where the clips (1) self-release as they are pushed off against the rim of the bone socket (20) (see FIG. 7).

An alternative embodiment is shown in FIGS. 8-11. This embodiment has secondary pressing positions (21) to release the clips (1) from the cup implant if necessary after the cup is fully impacted into the bone socket. Furthermore a latch (22) engages into a recess (23) to more firmly hold the clips in the secured position as can be seen in FIGS. 10-11.

Prior to use the holder (3) is engaged with the cup implant (8) by aligning the contoured flange (4) with corresponding contoured rim of the cup (6) which fits in one rotational position. As described above, the holder does not necessarily have to have a contoured flange that exactly corresponds to a contoured rim of the cup. The clips (1) are then pressed down over the outside edge of the cup by pressing on the surface grips (9) (if present) on top of the clips as can be seen from progression from FIG. 3 to FIG. 4 (and FIG. 8 to 9) also indicated by arrows A. Alternatively the holder is supplied with the implant in the same sterile pack with the clips in the manufactured position (as shown in FIG. 3) so that the surgeon can easily inspect the bearing surface (13). Once the holder is secured to the cup implant, a shaft (16) is inserted into the holder boss (2) as shown in FIG. 5. The shaft with assembled holder and cup implant is then used to position the cup implant in to the pre-machined bone socket (19) and impact it into place with a small interference fit as shown in FIG. 6. When fully impacted into the bone socket the clips either self-release as they press against the bone socket rim (20) or are released by pulling the holder away from the cup or are released by manually releasing the clips. Manual release of the clips is made easier by the addition of outward facing pressing pads (21) as shown in an alternative embodiment in FIGS. 8-11.

1. A holder for an implantable device, said holder comprising;
a body portion; and
at least one fastener attached to the body portion, wherein the at least one fastener is operable in order to secure said holder to said implantable device.

2. The holder of claim 1, wherein the body portion comprises;
an outer surface which is contactable to the implantable device; and
an inner surface, wherein the at least one fastener is attached to the inner surface.

3. The holder of claim 1, wherein the body portion of the holder has an outer surface that is profiled to complement an inner surface of the implantable device.

4. The holder of claim 1, wherein the at least one fastener comprises a clip, wherein the clip is attached to an inner surface of the body portion of the holder, and wherein the clip is arranged such that a portion of the clip extends beyond a periphery of the body portion of the holder.

5. (canceled)

6. The holder of claim 1, wherein the at least one fastener is attached to the body portion by a bridge of material, and wherein the bridge of material is resiliently biased such that a resting state of the at least one fastener is an open non-gripping configuration.

7. (canceled)

8. The holder of claim 1, wherein the at least one fastener comprises at least one latching member arranged to face an inner surface of the body portion of the holder, and wherein the body portion has a complementary recess to accept the latching member.

9. The holder of claim 1, wherein the at least one fastener is formed as an extension of the body portion.

10. The holder of claim 1, wherein the at least one fastener has a gripping portion at a distal region of the fastener, said gripping portion arranged to be in frictional contact with the outer surface of the implantable device when the at least fastener is operated to secure the holder to the implantable device.

11. The holder of claim 1, wherein the at least one fastener comprises at least one tab arranged such that moving said tab towards an axis of the holder raises a distal gripping portion of the at least one fastener.

12. (canceled)

13. The holder of claim 1, wherein the body portion of the holder comprises a centrally disposed boss.

14.-15. (canceled)

16. The holder of claim 13, wherein the body portion further comprises at least one additional structure connecting the inner surface of the body portion and the body, and wherein the at least one additional structure is at least one fin extending from the boss to the inner surface of the body portion.

17. (canceled)

18. The holder of claim 1, wherein the at least one fastener is manufactured with the body portion of the holder as a single component.

19. The holder of claim 1, wherein the holder is manufactured from plastic by additive manufacturing.

20. (canceled)

21. The holder of claim 1, wherein a peripheral edge of the body portion of the holder is contoured, and wherein the contour matches a contoured rim of the implantable device.

22. (canceled)

23. The holder of claim 1, wherein the implantable device is an acetabular cup implant, optionally with a large diameter bearing and optionally without specific cup holding features.

24. A system, comprising;
an implantable device; and
a holder, comprising:
a body portion; and
at least one fastener attached to the body portion, wherein the as least one fastener is operable in order to secure said holder to said implantable device.

25. (canceled)

26. The system of claim 24, wherein the at least one fastener is releasable from the implantable device by being pushed off against the rim of a bone socket as the implantable device is inserted into the bone socket, but remains attached to the holder.

27. A method, comprising:
inserting said holder into said implantable device such that an outer surface of the holder makes contact with an inner surface of said implantable device; and
operating said at least one fastener such that the holder is securely attached to said implantable device.

28.-29. (canceled)

30. The method of claim 27, further comprising:
inserting the implantable device with the attached holder into a bone socket; and
releasing the at least one fastener from the implantable device while retaining the at least one fastener attached to the holder by pushing the at least one fastener against the rim of the bone socket as the implantable device is inserted into the bone socket.

31. The method of claim 27, further comprising:
fixedly inserting the implantable device with the attached holder into a bone socket; and
releasing the at least one fastener from the implantable device while retaining the at least one fastener attached to the holder by pulling the holder away from the implantable device which is fixed into the bone socket.

32. (canceled)
